(19) United States
(12) Patent Application Publication
(10) Pub. No.: US 2025/0134710 A1
(43) Pub. Date: May 1, 2025

(54) PHACOEMULSIFICATION HANDPIECE WITH INTEGRATED ANTI-VACUUM SURGE MODULE

(71) Applicant: JOHNSON & JOHNSON SURGICAL VISION, INC., Irvine, CA (US)

(72) Inventors: Assaf Govari, Haifa (IL); Ilya Sitnitsky, Nahariya (IL); Sergey Sobol, Haifa (IL); Alexander Shechtman, Haifa (IL); Elad Avraham Diukman, Haifa (IL); Stanislav Katzir, Haifa (IL)

(21) Appl. No.: 18/383,665

(22) Filed: Oct. 25, 2023

(51) Int. Cl. A61F 9/007; A61M 1/00

(52) U.S. Cl. CPC A61F 9/00745; A61M 1/743; A61M 1/75; A61M 2210/0612

(57) ABSTRACT

A phacoemulsification probe includes a distal end, an aspiration channel, and an anti-vacuum surge (AVS) module. The distal end is configured for insertion into an eye of a patient. The aspiration channel is coupled with the distal end for evacuating material from the eye. The AVS module includes (i) a disposable portion, which is detachably coupled with the aspiration channel, and comprises a valve part configured to be moved to regulate flow in the aspiration channel, and (ii) a reusable portion, which is configured to move the valve part in response to one or more command signals.

### FIELD OF THE DISCLOSURE

The present disclosure relates generally to phacoemulsification systems and probes, and particularly to and particularly to systems for aspiration control.

### BACKGROUND OF THE DISCLOSURE

A cataract is a clouding and hardening of the eye's natural lens, a structure that is positioned behind the cornea, iris, and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then, a portion of the anterior surface of the lens capsule is removed, to gain access to the cataract. The surgeon then uses a phacoemulsification probe including an ultrasonic handpiece with a needle. The tip of the needle vibrates at an ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient's vision.

The present disclosure will be more fully understood from the following detailed description of the examples thereof, taken together with the drawings in which:

### BRIEF DESCRIPTION OF THE DRAWINGS

FIG. 1 is a schematic, pictorial view, along with an orthographic side view, of a phacoemulsification system comprising a phacoemulsification handpiece fitted with an integrated anti-vacuum surge (AVS) module, in accordance with an example of the present disclosure;

FIG. 2 is a schematic, orthographic side view of the rear portion of a phacoemulsification handpiece that includes an integrated AVS module, in accordance with an example of the present disclosure;

FIG. 3 is an exploded rendering of the integrated AVS module of FIG. 2, in accordance with some examples of the present disclosure; and

FIG. 4 is a flow chart schematically illustrating a method for performing a sterile phacoemulsification procedure on multiple patients using the same handpiece of FIG. 1 by replacing a disposable AVS element of the handpiece, in accordance with some examples of the present disclosure.

### DETAILED DESCRIPTION OF EXAMPLES

### Overview

During phacoemulsification, emulsified lens particles are aspirated using a phacoemulsification probe. When a particle blocks the inlet of the aspiration channel of the probe, the vacuum in the line increases. When the line later becomes unblocked (e.g., when the particle is subsequently sucked into the line), the high vacuum in the line causes a vacuum surge with potentially traumatic consequences to the eye.

One recent solution to the problem of vacuum surge is described in U.S. patent application Ser. No. 17/130,409, filed on Dec. 22, 2020, and titled, “A module for Aspiration and Irrigation Control,” which is assigned to the assignee of the present application. Application Ser. No. 17/130,409 discloses an anti-vacuum surge (AVS) standalone module coupled with a phacoemulsification probe, which prevents a sudden vacuum increase from being transferred to the eye when an occlusion breaks. In one example, the module can mitigate the vacuum surge by closing off a connection from the aspiration channel to the eye at the distal side of the module.

The standalone AVS module includes a valve that can be quickly activated when the onset of a vacuum surge is detected, typically a solenoid-type valve with a coil and magnet. However, the standalone AVS is a disposable device that is coupled with the handpiece near the proximal end of the handpiece. The added bulk and weight of the AVS module make the ergonomics of the handpiece less favorable. Furthermore, disposing of the entire standalone AVS device is both expensive and wasteful.

Examples of the present disclosure that are described hereinafter provide a handpiece with an internal integrated AVS module. In one example, the AVS module includes a portion comprising a small number of disposable parts. In another example, the entire AVS is reusable, i.e., the integrated AVS module includes no disposable parts and can withstand sterilization.

In the case of an integrated AVS module that includes a disposable portion, metallic parts of the AVS module are reusable, and the portion that is disposable is made of less expensive materials, e.g., plastic. The disposable portion includes a valve part (e.g., a rod) that extends into a channel through which the aspiration flow is directed. The rod includes a hole through which the aspiration flow can be directed when the hole is aligned with the channel. A connector on the head of the rod is coupled with the solenoid that rotates the rod to either an open or closed state. In another example, the rod traverses the aspiration channel (e.g., in a linear motion), between the hole being aligned with the channel and the channel being closed. Other implementations are also possible for achieving the same functionality.

The disposable portion of the AVS module is removed at the end of a current phacoemulsification procedure, and a new disposable portion is inserted into the handpiece before the beginning of the next phacoemulsification procedure.

The disclosed integrated AVS module is typically controlled by a processor of the phacoemulsification system. The processor controls the AVS module by using real-time readings received by the processor from sensors that are fluidly coupled with the aspiration channel and, optionally, with the irrigation channel of the handpiece.

### System Description

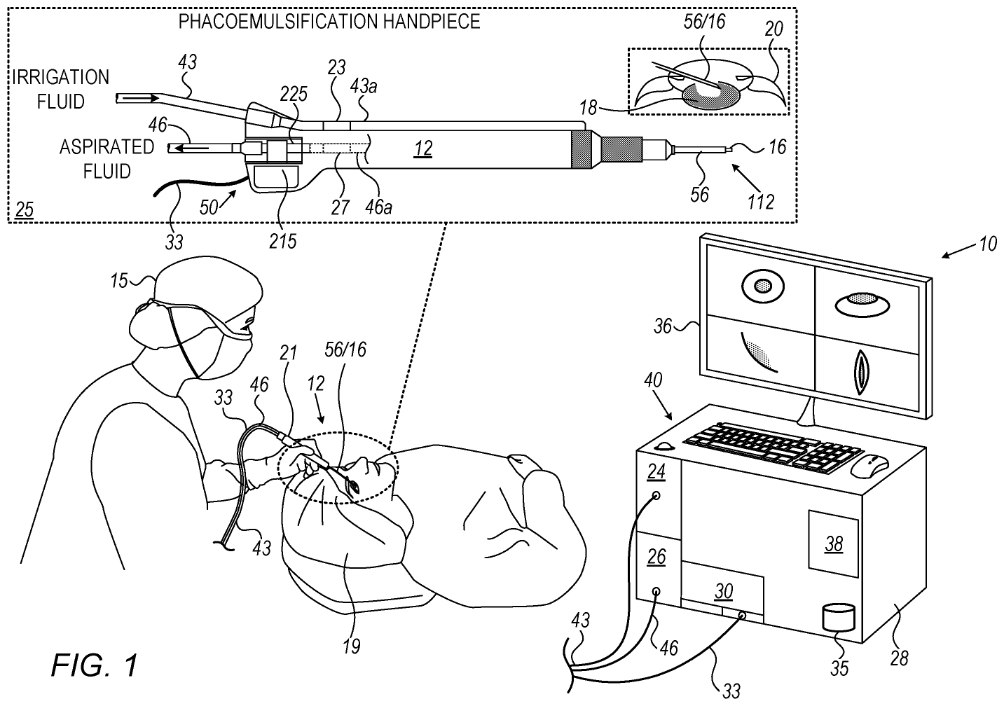

FIG. 1 is a schematic, pictorial view, along with an orthographic side view, of a phacoemulsification system 10 comprising a phacoemulsification handpiece 12 fitted with integrated anti-vacuum surge (AVS) module 50, in an accordance with an example of the present disclosure.

As seen in the pictorial view of phacoemulsification system 10, and in inset 25, a phacoemulsification probe 12 (e.g., handpiece 12) comprises a distal end 112 comprising a needle 16 and a coaxial irrigation sleeve 56 that at least partially surrounds needle 16. Sleeve 56 creates a fluid pathway between the external wall of the needle and the internal wall of the irrigation sleeve, while needle 16 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA.

In the shown example, during the phacoemulsification procedure a pumping subsystem 24, comprised in a console 28, pumps irrigation fluid from an irrigation reservoir (not shown) to the irrigation sleeve 56 to irrigate the eye. The fluid is pumped via an irrigation tubing line 43 running from console 28 to an irrigation channel 43a of probe 12. Eye fluid and waste matter (e.g., emulsified parts of the cataract) are aspirated via hollow needle 16 to a collection receptacle (not shown) by a pumping subsystem 26, also comprised in console 28, using an aspiration tubing line 46 running from aspiration channel 46a of probe 12 to console 28. In another example, the pumping subsystem 24 may be coupled with, or replaced by, a gravity-fed irrigation source, such as a balanced salt solution bottle/bag.

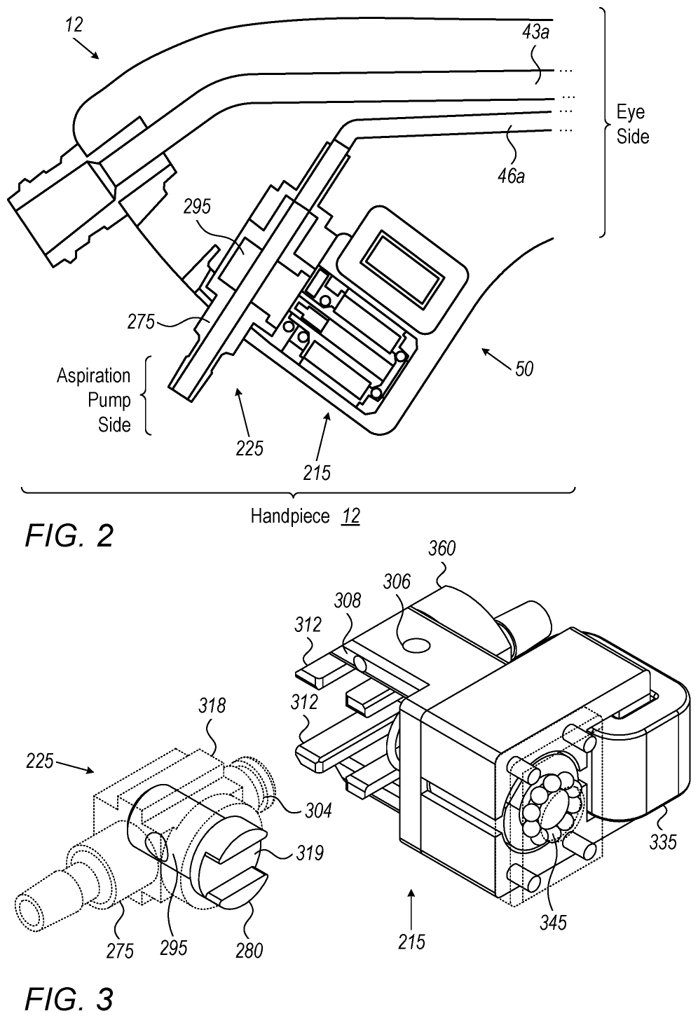

Handpiece 12 includes a standalone, partially disposable, AVS module 50 to control (e.g., regulate) aspiration flow to reduce risks to eye 20 from irregular performance of aspiration in probe 12, such as from a vacuum surge. AVS module 50 comprises a fixed portion 215 that can undergo sterilization, and a disposable portion 225 that is replaced between phacoemulsification procedures, as described in FIGS. 2 and 3.

Module 50 can discontinue aspiration to provide a fast response (e.g., within several milliseconds) to a detected vacuum surge. Module 50 is typically commanded by processor 38 via a cable 33. In the shown example, fast and robust control over intraocular pressure (IOP) during a surgical cataract removal procedure is facilitated by module 50. Processor 38 controls AVS module 50 using real-time readings received by processor 38 from sensor arrays 23 and 27. Sensor array 23 comprises a number of pressure sensors (e.g., three) that are fluidly coupled with irrigation channel 43a. Sensor array 27 comprises an odd number of vacuum sensors (e.g., three or five) that are fluidly coupled with aspiration channel 46a. An example of such a sensor array is described in U.S. patent application Ser. No. 18/089, 399, filed Dec. 27, 2022, and titled, “Pressure Sensing Array in Phacoemulsification Handpiece,” which is assigned to the assignee of the present application.

Phacoemulsification probe 12 includes other elements (not shown), such as one or more piezoelectric crystals coupled with a horn to drive the vibration of needle 16. The piezoelectric crystal is configured to vibrate needle 16 in a resonant vibration mode. The vibration of needle 16 is used to break a cataract into small pieces during a phacoemulsification procedure. Console 28 comprises a piezoelectric drive module 30, coupled with the piezoelectric crystal, using electrical wiring running in cable 33. Drive module 30 is controlled by a processor 38 and conveys processor-controlled driving signals via cable 33 to, for example, maintain needle 16 at a selected vibration amplitude. The drive module may be realized in hardware or software, for example, in a proportional-integral-derivative (PID) control architecture.

Processor 38 may receive user-based commands via a user interface 40, which may include setting a vibration mode, duty cycle, and/or frequency of the piezoelectric crystal, and setting or adjusting an irrigation and/or aspiration rate of the pumping subsystems 24/26. In one example, user interface 40 and display 36 may be combined as a single touchscreen graphical user interface. In another example, the physician uses a foot pedal (not shown) as a means of control, and an encoder sensing the position of the foot pedal may provide input to processor 38. Additionally, or alternatively, processor 38 may receive user-based commands from controls located in handpiece 12.

Some or all of the functions of processor 38 may be combined in a single physical component or, alternatively, implemented using multiple physical components. These physical components may comprise hard-wired or programmable devices or a combination thereof. In some examples, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 may include further elements which are omitted for clarity of presentation. For example, physician 15 typically performs the procedure using a stereomicroscope or magnifying glasses, neither of which are shown. Physician 15 may use other surgical tools in addition to probe 12, which are also not shown to maintain clarity and simplicity of presentation.

In some examples, a different type of AVS module can be used that is coupled only with the aspiration part of the system (i.e., without involving irrigation).

### Phacoemulsification Handpiece Including an Integrated AVS Module Comprising a Disposable Part

FIG. 2 is a schematic, orthographic side view of the rear portion of phacoemulsification handpiece 12 including the integrated AVS module 50, in accordance with an example of the present disclosure. In the example shown in FIG. 2, a disposable protection valve portion 225 of module 50 can regulate aspiration flow based on vacuum readings from sensor array 27. Reusable portion 215 includes a driver and coupling mechanism portion 215 to rotate the valve part 295 (e.g., a rod 295) of disposable part 225 to open or close an aspiration channel going through 275 of disposable protection valve portion 225, as further described in FIG. 3.

The handpiece shown in FIG. 2 is simplified for clarity. It may include further elements that are omitted for simplicity of presentation, such as an inset detailing sensor arrays 23 and 27.

FIG. 3 is an exploded rendering of the integrated AVS module 50 of FIG. 2, in accordance with some examples of the present disclosure.

As seen, integrated AVS module 50 includes a reusable portion 215 comprising a solenoid-type mechanism for a rotating valve with a coil 335 and magnet 345. Further, reusable portion 215 includes a housing 360 to accommodate the detachable disposable protection valve portion 225. A mechanical coupling element (not seen) transfers a rotation of the magnet 345 to a rotating rod 295 to either an open or closed state.

The rod 295 extends into a channel of fixed housing 275 of the disposable protection valve portion 225 through which the aspiration flow is directed. The rod 295 includes a hole through which the aspiration flow can be directed when the hole is aligned with the channel. A connector 280 on the head of rod 295 is mechanically coupled (e.g., using a screw head geometry 319) with the solenoid for rotating the rod to either an open or closed state.

Disposable part 225 is readily inserted and accurately placed into housing 360 of the reusable portion 215 using rails 312 for mechanical guide, and an alignment assembly The combination of these mechanical elements (318). ensures that the rotor of magnet 345 and rotating rod 295 are well aligned. An O-ring (304) of the disposable portion mates with the reusable portion to ensure a sealed fluid connection. The entire disposable part 225 is locked in place by a spring-loaded mechanism including a release button 306 that a user can use to release the disposable part after use. Optionally, a lever may be used instead of a release button 309.

### Method for Performing Sterile Phacoemulsification Procedures Using a Handpiece Comprising a Disposable AVS Potion

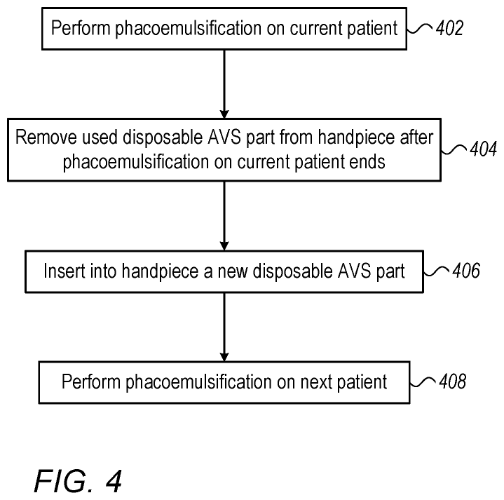

FIG. 4 is a flow chart schematically illustrating a method for performing a sterile phacoemulsification procedure on multiple patients using the same handpiece 12 of FIG. 1 by replacing a disposable AVS portion 225 of the handpiece 12, in accordance with some examples of the present disclosure.

The process begins with physician 15 performing phacoemulsification on a patient using handpiece 12 comprising AVS module 50, as seen in FIG. 1, at a current phacoemulsification step 402.

At an AVS portion removal step 404, after the phacoemulsification on the current patient has ended, a user (e.g., a medical team member) removes the disposable AVS portion 225 from handpiece 12.

The surgeon has a set of sterilizable handpieces and a new one is used for each patient. (At some point in the day or at the end of the day the used handpieces are sterilized.)

In an AVS portion insertion step 406, the medical team member inserts a new disposable AVS portion 225 into handpiece 12. Finally, at a new phacoemulsification step 408, physician 15 performs phacoemulsification on a new patient using handpiece 12 comprising the renewed AVS module 50.

### EXAMPLES

### Example 1

A phacoemulsification probe (12) includes a distal end (112), an aspiration channel (46a), and an anti-vacuum surge (AVS) module (50). The distal end (112) is configured for insertion into an eye (20) of a patient (19). The aspiration channel (46a) is coupled with the distal end (112) for evacuating material from the eye (20). The AVS module (50) includes (i) a disposable portion (225), which is detachably coupled with the aspiration channel (46a), and comprises a valve part (285) configured to be moved to regulate flow in the aspiration channel (46a), and (ii) a reusable portion (215), which is configured to move the valve part (295) in response to one or more command signals.

### Example 2

The probe (12) according to example 1, wherein the valve part (295) is configured to be rotated between an open state and a closed state.

### Example 3

The probe (12) according to any of examples 1 and 2, wherein the one or more command signals are generated by a processor (38) of a phacoemulsification system.

### Example 4

The probe (12) according to any of examples 1 through 3, wherein the disposable portion (225) is made of plastic materials.

### Example 5

The probe (12) according to any of examples 1 through 4, wherein the disposable portion (225) is locked inside a housing portion (360) of the reusable portion (215) by a spring-loaded mechanism.

### Example 6

A phacoemulsification method includes inserting a distal end (112) of a phacoemulsification probe (12) into an eye of a patient. Material is evacuated from the eye using an aspiration channel (46a) coupled with the distal end (112). Flow in the aspiration channel (46a) is regulated using an anti-vacuum surge (AVS) module (50) inside the phacoemulsification probe (12), the AVS (50) comprising (i) a disposable portion (225), which is detachably coupled with the aspiration channel (46a), and comprises a valve part (295) configured to be moved to regulate the flow in the aspiration channel (46a), and (ii) a reusable portion (215), which is configured to move the valve part (295) in response to one or more command signals.

It will be appreciated that the examples described above are cited by way of example and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A phacoemulsification probe, comprising:
a distal end configured for insertion into an eye of a patient;
an aspiration channel coupled with the distal end for evacuating material from the eye; and
an anti-vacuum surge (AVS) module comprising:
a disposable portion, which is detachably coupled with the aspiration channel, and comprises a valve part configured to be moved to regulate flow in the aspiration channel; and
a reusable portion, which is configured to move the valve part in response to one or more command signals.

2. The probe according to claim 1, wherein the valve part is configured to be rotated between an open state and a closed state.

3. The probe according to claim 1, wherein the one or more command signals are generated by a processor of a phacoemulsification system.

4. The probe according to claim 1, wherein the disposable portion is made of plastic materials.

5. The probe according to claim 1, wherein the disposable portion is locked inside a housing portion of the reusable portion by a spring-loaded mechanism.

6. A phacoemulsification method, comprising:
inserting a distal end of a phacoemulsification probe into an eye of a patient;
evacuating material from the eye using an aspiration channel coupled with the distal end; and
regulating flow in the aspiration channel using an anti-vacuum surge (AVS) module inside the phacoemulsification probe, the AVS comprising:
a disposable portion, which is detachably coupled with the aspiration channel, and comprises a valve part configured to be moved to regulate the flow in the aspiration channel; and
a reusable portion, which is configured to move the valve part in response to one or more command signals.

7. The method according to claim 6, wherein the valve part is configured to be rotated between an open state and a closed state.

8. The method according to claim 6, wherein the one or more command signals are generated by a processor of a phacoemulsification system.

9. The method according claim 6, wherein the disposable portion is made of plastic materials.

10. The method according to claim 6, wherein the disposable portion is locked inside a housing portion of the reusable portion by a spring-loaded mechanism.
